(19) United States
(12) Patent Application Publication
(10) Pub. No.: US 2007/0032844 A1
(43) Pub. Date: Feb. 8, 2007

(54) Targeted UV phototherapy light block

(76) Inventor: Jeffrey I. Levatter, Solana Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/499,609

(22) Filed: Aug. 4, 2006

Related U.S. Application Data
(60) Provisional application No. 60/705,838, filed on Aug. 5, 2005.

(51) Int. Cl. A61B 18/18

(52) U.S. Cl. 607/88; 606/9

(57) ABSTRACT

A method of treating an epidermal region including diseased tissue and non-diseased tissue includes coating the non-diseased tissue with a substance that substantially attenuates ultraviolet light when coated onto the non-diseased tissue and illuminating the diseased tissue with ultraviolet light.

### CROSS-REFERENCE TO RELATED APPLICATIONS

This application claims priority under 35 U.S.C. § 119(e) to U.S. Provisional Application Ser. No. 60/705,838, filed Aug. 5, 2005, entitled “UV Light Attenuator,” which is incorporated herein by reference in its entirety.

### BACKGROUND

1. Field

The present invention relates to a method for treating skin disorders. In particular, the present invention relates to a method for treating skin disorders involving exposing a patient's skin to high intensity ultraviolet (UV) light.

2. Description of the Related Art

Ultraviolet light may be employed to treat a variety of skin disorders such as, for example, psoriasis and vitiligo. High doses of narrow band ultraviolet light directed onto a localized region of the skin has been shown to effectively treat such disorders with reduced risk of side affects such as severe radiation bums and skin cancer. Exemplary systems and methods for providing such treatment are described in U.S. patent application Ser. No. 10/799,337, entitled “Treatment of Skin Disorders with UV Light and Cooling” published at U.S. Patent Publication No. 2005/0015124 A1, now U.S. Pat. No. ______, which is incorporated herein by reference it its entirety. Such systems may include an ultraviolet light source and a delivery system.

In some systems, for example, excimers in a chamber of an excimer laser may be used to generate laser energy at a wavelength of about 308 nanometers (nm). The laser energy can be coupled from the laser chamber and delivered to the treatment site by using a flexible or rigid optical line such as a fiberoptic cable or liquid light guide or a delivery system including one or more mirrors. A wide variety of systems are possible.

As described above, to reduce harmful side affects such as severe radiation bum and cancer, the UV light exposure is limited. What is desired, therefore, are methods of focusing the UV light onto localized regions of diseased tissue.

### SUMMARY

In certain embodiments, a method of treating an epidermal region comprising diseased tissue and non-diseased tissue comprises coating the non-diseased tissue with a substance that substantially attenuates ultraviolet light when coated onto the non-diseased tissue and illuminating the diseased tissue with ultraviolet light.

### BRIEF DESCRIPTION OF THE DRAWINGS

FIG. 1 depicts an area of tissue including diseased and healthy regions amenable for treatment with UV light.

### DETAILED DESCRIPTION OF THE PREFERRED EMBODIMENTS

As described above and in U.S. patent application Ser. No. 10/799,337, entitled “Treatment of Skin Disorders with UV Light and Cooling,” published at U.S. Patent Publication No. 2005/0015124 A1, now U.S. Pat. No. ______, which is incorporated herein by reference it its entirety, ultraviolet light may be employed to treat a variety of skin disorders such as, for example, psoriasis and vitiligo. High doses of narrow band ultraviolet light directed onto a localized region of the skin has been shown to effectively treat such disorders with reduced risk of side affects such as severe radiation bums and skin cancer.

In various preferred embodiments, for example, narrow band UV light at 308 nanometers (“UV B” radiation) directed onto a diseased area of tissue to be treated has a high intensity, or “fluence,” for example about 100 millijoules per square centimeter (mJ/cm²) or greater. Given the intensity level of this irradiation, a clinician may wish to screen healthy tissue from the radiation in order to reduce the risk of damaging healthy tissue with the UV light.

Accordingly, in many cases, a form of shielding template, which is opaque to the UV radiation and having openings of varying sizes and shapes to allow UV light to pass, is used to shield healthy tissue from high intensity UV light. A portion of the template is superimposed on the periphery of the treatment site. The healthcare provider situates the template such that diseased tissue is not blocked by the template. Once a portion of the site has been treated with UV light, the template is moved around to find a near fit to the next portion of the periphery of the treatment site (such as finding a fit for a piece of a jigsaw puzzle). Given that some treatment sites are more three-dimensional than planar, fitting the template to a site can be a test of a clinician's spatial ingenuity. Colloquially, such templates are called “photobunnies,” but the jocular name merely conceals the effort and care that goes into the use of these templates.

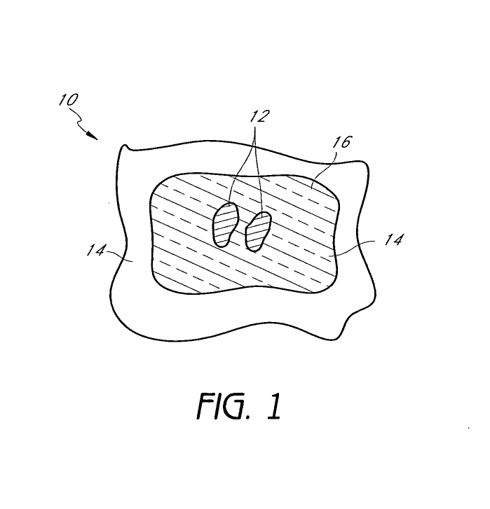

FIG. 1 shows a region of epidermal tissue 10 that includes diseased or “affected” tissue areas 12 and non-diseased or “healthy” tissue areas 14. In various preferred embodiments of the invention, a substance that attenuates or substantially attenuates the transmission of UV light is applied on the healthy areas 16 of the tissue 10. In some embodiments, the substance attenuates the transmission of UV light after being coated on epidermal tissue, for example after drying. After the coating, the diseased tissue 12 is illuminated with ultraviolet light to treat the diseased tissue 12. As used herein, the term “coat” is to be given its broadest possible meaning, including, but not limited to, to partially cover a surface, to substantially cover a surface, and to fully cover a surface.

In certain embodiments, the ultraviolet light has a wavelength between about 300 and 315 nm (e.g., of about 308 nm). Narrow band ultraviolet light having a band of between about 300 and 315 nm or smaller, for example, between about 305 and 310 nanometers or between about 307 and 309 nanometers may be used in certain embodiments. The narrow band of light may be centered at 308 nm and have a bandwidth of, e.g., ±7 nm, 5 nm, 3 nm, 1 nm, or less. Other bands of UV light may also be used.

The intensity of the ultraviolet light may be greater than about 1 minimum erythema dose (MED), greater than about 5 MED, greater than about 10 MED, greater than about 20 MED, etc. The intensity of the ultraviolet light at 308 nm may be greater than about 100 mJ/cm², greater than about 500 mJ/cm², greater than about 1,000 mJ/cm², greater than about 2,000 mJ/cm², etc.

Various embodiments described herein relate to blocking ultraviolet light from skin or other tissue that is preferably not to be treated with UV light. In some embodiments, such light originates from a light source such as a laser (e.g., an excimer laser) or other type of light source (e.g., a UV lamp).

In certain embodiments, the substance comprises a fluid (e.g., cream, lotion, ointment, sun-block), a powder, a paste, and the like. The substance may be selected based on a variety of factors, including, but not limited to, the disease to be treated, the intensity of UV light to be used, amenability to the patient, and the like. For example, a cream can be particularly useful in the treatment of vitiligo, where disfiguration of the face can be especially stigmatizing and where care is taken to not worsen the disfigurement. Other substances that block UV light, both well-known in the art as well as substances yet to be devised, may be employed. Such UV blocking substances may be applied to the patient, for example, in a manner so as to outline the periphery of the treatment site.

In certain preferred embodiments, the substance is easy to apply to the patient's skin. Other characteristics of the substance may be varied, such as viscosity, color, smell, taste, etc. For example, the substance may be clearly visible when applied to assist the healthcare provider in identifying portions of the tissue covered by the substance. In some embodiments, however, the substance may appear clear to the practitioner and may be substantially non-transmissive to UV light. Other characteristics of this substance may be varied. In some embodiments, the substance is partially transmissive of UV B light. The substance may, however, transmit not more than 50%, 30%, 20%, 10%, or less UV light of the wavelength band used to illuminate and treat the diseased tissue. Accordingly, the substance may block 50%, 70%, 80%, 90%, or more of the UV light directed onto the diseased tissue. Some examples of substances that may be used include without limitation, BullFrog® Superblock Lotion, available from Chattem, Inc. of Chattanooga, Tenn. In certain embodiments, a plurality of substances are coated on the non-diseased tissue.

In various preferred embodiments, the substance comprises a composition that blocks a wide range of wavelengths including wavelengths outside the UV range. For example, a paste comprising opaque particulates may be employed. In certain embodiments in which the wavelength of the UV light is known, the substance may be selected based on wavelength of the UV light. In certain embodiments in which the intensity of the UV light is known, the substance may be selected based on intensity of the UV light. In general, the more intense the dosage of UV light, the higher should be the SPF (“sun protection factor”) used. Other compositions are possible and should not be limited to those specifically recited herein.

The substance may absorb and/or reflect the UV light. The substance may be used in combination with a template (e.g., by screening a portion of the non-diseased tissue from the UV light with the template), or may be employed without the use of templates. Rigid or flexible templates may be employed.

As described above, the substance is applied to the patient on healthy tissue that is preferably exposed to reduced levels of UV light. The diseased tissue may be exposed to high dosages of the UV light for treatment while tissue to which the substance is applied receives a reduced and preferably a negligible dosage of such UV light. Risk of damage to healthy tissue, which may be adjacent to the diseased tissue, may thereby be reduced. As described above, the blocking agents can be employed with laser or non-laser light sources and delivery systems that are used in the treatment of various disorders such as skin disorders like psoriasis and others. In some embodiments, the agent may be supplied together with the UV light source, accessories (e.g., templates), or supplies for such a system or for such treatment in a kit. In some embodiments, the UV attenuating substance may be provided separately.

A wide variety of variations are possible. For example, processing steps may be added or removed, or reordered.

While the foregoing detailed description discloses several embodiments of the present invention, it should be understood that this disclosure is illustrative only and is not limiting of the present invention. It should be appreciated that the specific configurations and operations disclosed can differ from those described above, and that the methods described herein can be used in other contexts.

What is claimed is:

1. A method of treating an epidermal region comprising diseased tissue and non-diseased tissue, the method comprising:
coating the non-diseased tissue with a substance that substantially attenuates ultraviolet light when coated onto the non-diseased tissue; and
illuminating the diseased tissue with ultraviolet light.

2. The method of claim 1, wherein said substance comprises a fluid.

3. The method of claim 2, wherein the fluid comprises a cream, lotion, or ointment.

4. The method of claim 1, wherein the substance comprises a powder.

5. The method of claim 1, wherein the substance comprises a paste.

6. The method of claim 5, wherein the paste comprises opaque particulates.

7. The method of claim 1, wherein the substance comprises sun-block.

8. The method of claim 1, wherein the substance is visible when coated onto the non-diseased tissue.

9. The method of claim 1, wherein said illuminating comprises illuminating said diseased tissue with narrow band ultraviolet light between about 300 and 315 nm.

10. The method of claim 9, wherein said illuminating comprises illuminating said diseased tissue with narrow band ultraviolet light having a central wavelength of about 308 nanometers.

11. The method of claim 1, wherein the illuminating comprises directing UV light having an intensity greater than about 1 minimum erythema dose (MED) onto the epidermal region.

12. The method of claim 11, wherein the illuminating comprises directing UV light having an intensity greater than about 5 MEDs onto the epidermal region.

13. The method of claim 1, wherein the illuminating comprises directing ultraviolet light having an intensity greater than about 100 mJ/cm² onto the diseased tissue.

14. The method of claim 1, wherein the coating comprises coating a plurality of substances onto the non-diseased tissue.

15. The method of claim 1, further comprising producing said ultraviolet light with a laser.

16. The method of claim 15, wherein said laser comprises an excimer laser.

17. The method of claim 1, further comprising producing said ultraviolet light with a UV lamp.

18. The method of claim 1, further comprising selecting a fluid based on the wavelength of the ultraviolet light.

19. The method of claim 1, further comprising selecting a fluid based on the intensity of the ultraviolet light.

20. The method of claim 1, further comprising screening a portion of the non-diseased tissue from the UV light with a template.
